(19) United States
(12) Patent Application Publication
(10) Pub. No.: US 2008/0241875 A1
(43) Pub. Date: Oct. 2, 2008

(54) APPARATUS AND METHOD OF DETECTING MICROORGANISM OR MICRO-PARTICLE IN REAL TIME

(75) Inventors: Jung Joo HWANG, Suwon-si (KR); Mi Jeong SONG, Suwon-si (KR); Jin Sang HWANG, Suwon-si (KR); Jang Seok MA, Seongnam-si (KR)

Correspondence Address: CANTOR COLBURN, LLP, 20 Church Street, 22nd Floor, Hartford, CT, 06103, US

(73) Assignee: SAMSUNG ELECTRONICS CO., LTD, Suwon-si (KR)

(21) Appl. No.: 11/780,630

(22) Filed: Jul. 20, 2007

(30) Foreign Application Priority Data
Jan. 18, 2007 (KR) 10-2007-0005722

(51) Int. Cl. C12Q 1/06; C12M 1/00

(52) U.S. Cl. 435/39; 435/287.1

(57) ABSTRACT

An apparatus for detecting a microorganism or micro-particle in real time includes a collection module comprising a condensation element unit which condenses water particles in an atmosphere and forms a droplet to which a microorganism or micro-particle in the atmosphere adheres to, and a collection channel unit which gathers the droplet and generates a droplet stream and a sensing module including a counting module. The droplet stream is introduced to the sensing module, and the sensing module detects and counts the microorganism or micro-particle which is adhered to the introduced droplet stream.

### CROSS-REFERENCE TO RELATED APPLICATION

This application claims priority to Korean Patent Application No. 10-2007-0005722, filed on Jan. 18, 2007, and all the benefits accruing therefrom under 35 U.S.C. § 119, the contents of which in its entirety are herein incorporated by reference.

### BACKGROUND OF THE INVENTION

1. Field of the Invention

The present invention relates to an apparatus and method of detecting a microorganism or micro-particle, and more particularly, to an apparatus and method of detecting a microorganism or micro-particle in real time without an additional complex and expensive device.

2. Description of Related Art

Numerous microorganisms such as bacteria, viruses and molds float in the air, and humans are directly or indirectly affected by these microorganisms. Currently, many people have become interested in clean air and are aware of air pollution caused by microorganisms. Accordingly, a necessity of developing microorganism detection technologies which enable average people to easily monitor microorganisms at any place, in real time, has increased. Consumers' needs for an apparatus with such technologies have also increased.

The above-described apparatus which uses a conventional microorganism measurement method requires culturing for at least a few hours to a few days in a culture medium. The above-described apparatus may also be used with a professional analysis method, which is performed in a wet environment, e.g., polymerase chain reaction (“PCR”), an antigen-antibody reaction.

In order to measure microorganisms in the air, collecting samples for testing and for measuring an amount of or types of microorganisms from the collected samples are required. However, the conventional microorganism measurement method requires a significant amount of time and effort, and requires additional time to culture the collected samples.

In a culture method, samples are directly cultured in a culture medium, the number of formed colonies is counted, and thus a result of whether microorganisms exist within the collected samples is determined. However, more than 24 hours are typically expended in culturing the samples, and a period of over seven days is needed to detect fungi. Therefore, microorganisms may not be detected quickly enough.

In addition, since a few types of bacteria may not be cultured by typical methods, certain microorganisms within the collected samples may not be detected. Moreover, colony formation may be affected by several factors such as types of microorganisms, formation of the culture medium, time allowed for a culture, temperature, humidity and the like. Thus, the conventional microorganism measurement methods described above require a culture operation for acquiring several samples from the collected samples of the microorganisms. In addition, microorganisms which may be measured are limited to only those types of microorganisms which may be cultured.

An optical detection method acquires cell information by a light scattering phenomenon and light emitting phenomenon, or by a light emitting phenomenon of cells themselves in particular wavelengths. The light scattering phenomenon and light emitting phenomenon are generated when lasers pass through cells which are combined with fluorescent substances. The optical detection method is capable of rapidly and accurately analyzing the cells. However, combining cells with fluorescent substances causes unexpected changes to the cells. Further, an analysis apparatus used in the optical detection method is complex and expensive, and may not be easily moved or operated.

Currently, a microorganism measurement method based on an electrical method has been developed. However, research on an advanced collection operation, which applies an electrical method, has not been developed. Furthermore, the current electrical method typically requires liquid samples, and thus may not be suitable for the measurement of microorganisms. Thus, an apparatus and method of detecting a microorganism or micro-particle in real time is required.

### BRIEF SUMMARY OF THE INVENTION

An exemplary embodiment of the present invention provides an apparatus and method of detecting a microorganism or micro-particle in real time which may gather and sense the microorganism or micro-particle without a complex and expensive apparatus.

An exemplary embodiment of the present invention also provides an apparatus and method of detecting a microorganism or micro-particle in real time which does not require an additional conversion of an electrical signal.

An exemplary embodiment of the present invention also provides an apparatus and method of detecting a microorganism or micro-particle in real time which is based on a wet measurement and does not require an additional apparatus for providing water or other liquids.

According to an exemplary embodiment of the present invention, there is provided an apparatus for detecting a microorganism or micro-particle in real time, the apparatus includes a collection module comprising a condensation element unit which condenses water (“H₂O”) particles in an atmosphere and which forms a droplet to which a microorganism or micro-particle in the atmosphere is adhered to, and a collection channel unit which gathers the droplet and generates a droplet stream and a sensing module comprising a counting module, the sensing module in fluid communication with the droplet stream, and the sensing module detects and counts the microorganism or micro-particle which is adhered to the droplet stream.

According to another exemplary embodiment of the present invention, there is provided a method of detecting a microorganism or micro-particle in real time, the method includes condensing water (“H₂O”) particles in an atmosphere via a collection channel unit, forming a droplet from the condensed water particles to which a microorganism or micro-particle in the atmosphere is adhered to, gathering the droplet and generating a droplet stream via a collection channel unit, the collection channel unit comprising a hydrophilic material and introducing the droplet stream via a predetermined inflow unit, and detecting and counting the microorganism or micro-particle which is adhered to the introduced droplet stream via a Coulter counter.

According to another exemplary embodiment of the present invention, there is provided a collection module including a condensation element unit which condenses water particles in an atmosphere and which forms a droplet to which a microorganism or micro-particle in the atmosphere is adhered to and a collection channel unit which gathers the droplet and generates a droplet stream.

Additional aspects, features and/or advantages of the invention will be set forth in part in the description which follows and, in part, will be apparent from the description, or may be learned by practice of the present invention.

### BRIEF DESCRIPTION OF THE DRAWINGS

The above and/or other aspects, features and advantages of the present invention will now become apparent and more readily appreciated from the following description of exemplary embodiments, taken in conjunction with the accompanying drawings, in which:

FIG. 1 is a block diagram illustrating a configuration of an exemplary embodiment of an apparatus for detecting a microorganism or micro-particle in real time according to the present invention;

FIG. 2 is a front perspective cross-sectional view with a partial cutout illustrating an exemplary embodiment of an apparatus for detecting a microorganism or micro-particle in real time according to the present invention;

FIGS. 3A and 3B are a cross-sectional side view and a cross-sectional front view, respectively, of an exemplary embodiment of a collection module according to the present invention;

FIG. 4 is a cross-sectional view of an exemplary embodiment of a sensing module according to the present invention;

FIG. 5 is a pulse waveform diagram of a current change measured in an exemplary embodiment of a sensing module according to the present invention; and

FIG. 6 is a flowchart illustrating an exemplary embodiment of a method of detecting a microorganism or micro-particle in real time according to the present invention.

### DETAILED DESCRIPTION OF THE INVENTION

The invention now will be described more fully hereinafter with reference to the accompanying drawings, in which embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art.

It will be understood that when an element is referred to as being “on” another element, it can be directly on the other element or intervening elements may be present therebetween. In contrast, when an element is referred to as being “directly on” another element, there are no intervening elements present. As used herein, the term “and/or” includes any and all combinations of one or more of the associated listed items.

It will be understood that, although the terms first, second, third etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below could be termed a second element, component, region, layer or section without departing from the teachings of the present invention.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Furthermore, relative terms, such as “lower” or “bottom” and “upper” or “top,” may be used herein to describe one element's relationship to another elements as illustrated in the Figures. It will be understood that relative terms are intended to encompass different orientations of the device in addition to the orientation depicted in the Figures. For example, if the device in one of the figures is turned over, elements described as being on the “lower” side of other elements would then be oriented on “upper” sides of the other elements. The exemplary term “lower”, can therefore, encompasses both an orientation of “lower” and “upper,” depending on the particular orientation of the figure. Similarly, if the device in one of the figures is turned over, elements described as “below” or “beneath” other elements would then be oriented “above” the other elements. The exemplary terms “below” or “beneath” can, therefore, encompass both an orientation of above and below.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

Exemplary embodiments of the present invention are described herein with reference to cross section illustrations that are schematic illustrations of idealized embodiments of the present invention. As such, variations from the shapes of the illustrations as a result, for example, of manufacturing techniques and/or tolerances, are to be expected. Thus, embodiments of the present invention should not be construed as limited to the particular shapes of regions illustrated herein but are to include deviations in shapes that result, for example, from manufacturing. For example, a region illustrated or described as flat may, typically, have rough and/or nonlinear features. Moreover, sharp angles that are illustrated may be rounded. Thus, the regions illustrated in the figures are schematic in nature and their shapes are not intended to illustrate the precise shape of a region and are not intended to limit the scope of the present invention.

Reference will now be made in more detail to exemplary embodiments of the present invention, examples of which are illustrated in the accompanying drawings, wherein like reference numerals refer to the like elements throughout. Exemplary embodiments are described below to explain the present invention by referring to the figures.

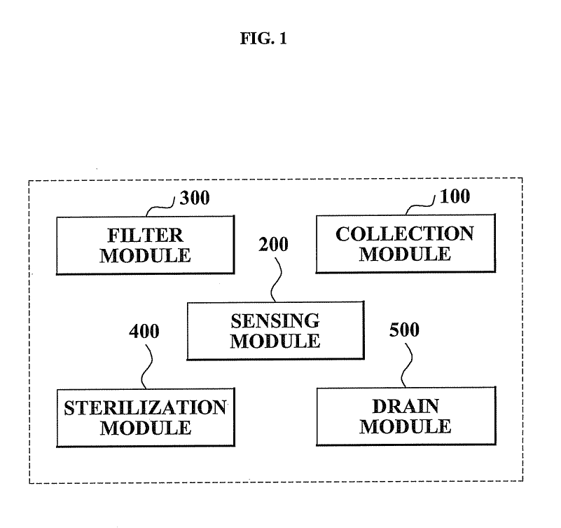

FIG. 1 is a block diagram illustrating a configuration of an exemplary embodiment of an apparatus for detecting a microorganism or micro-particle in real time according to the present invention.

Referring to FIG. 1, the apparatus for detecting a microorganism or micro-particle in real time includes a collection module 100, a sensing module 200, a filter module 300, a sterilization module 400 and a drain module 500.

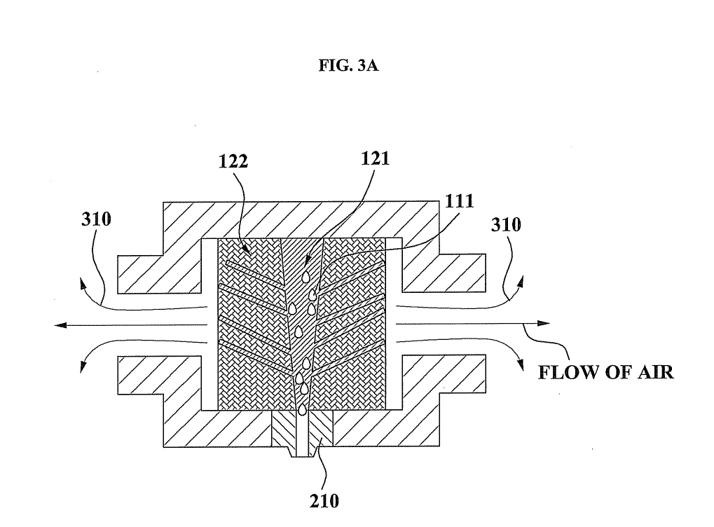

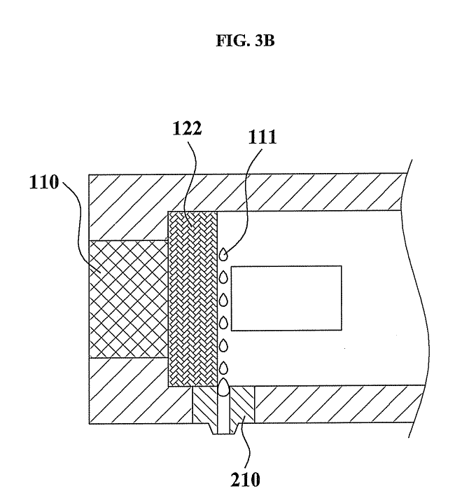

The collection module 100 condenses water (“H₂O”) particles in an atmosphere 310, and forms a droplet 111 to which a microorganism or micro-particle in the atmosphere 310 is adheres to. Also, the collection module 100 gathers the droplet 111 and generates a droplet stream. For this, the collection module 100 includes a condensation element unit 110 for condensing the H₂O particles in the atmosphere 310, and a collection channel unit 120 for gathering the droplet 111 and generating the droplet stream. The collection channel unit 110 includes a pattern where a hydrophilic member 121, which is in a vein structure, is formed in a hydrophobic member 122. Since the droplet 111 is comprised of a polarized molecule, the microorganism or micro-particle is electrically attracted to and adheres to the droplet 111. Also, the collection channel unit 110 structurally and functionally gathers and flocculates the droplet 111, and thereby may form the droplet stream. The collection channel unit 110 is described in more detail with reference to FIG. 3A.

The droplet stream is introduced to the sensing module 200 which detects and counts the microorganism or micro-particle which is adhered to the introduced droplet stream. For this, the sensing module 200 includes an inflow unit for introducing the droplet stream, and a Coulter counter for detecting and counting the microorganism or micro-particle which is adhered to the introduced droplet stream. In exemplary embodiments, the inflow unit may include a channel at a side of the Coulter counter, whereby the droplet stream passes through the channel. A width of the channel becomes gradually narrower in order to smoothly introduce the droplet stream which is adhered to the microorganism or micro-particle into the Coulter counter.

The filter module 300 filters out a microorganism or micro-particle which is larger than a predetermined size from the atmosphere 310, and guides the filtered microorganism or micro-particle to the collection module 100. In exemplary embodiments, the filter module 300 which filters the microorganism or micro-particle may include a pore size between about 0.5 μm and about 10 μm.

The sterilization module 400 sterilizes the detected and counted microorganism or micro-particle. The drain module 500 vaporizes the droplet stream by heating and drains a vaporized droplet stream via an outlet 520.

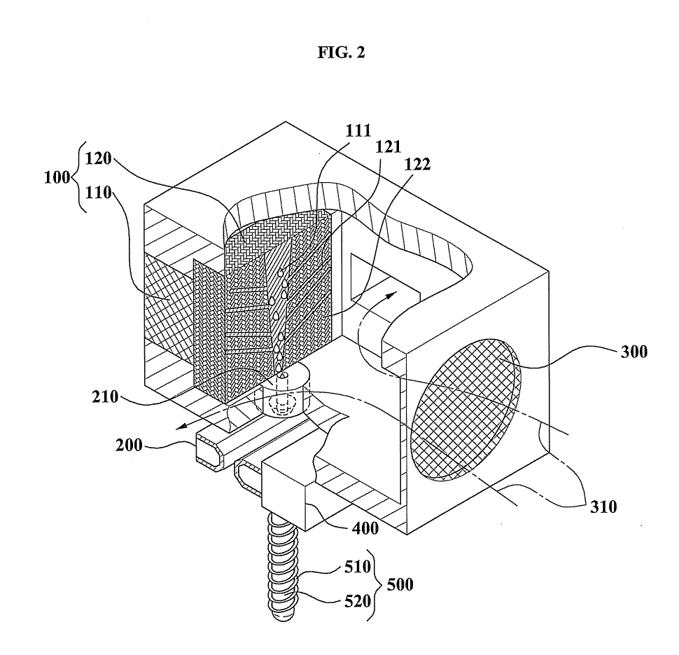

FIG. 2 is a front perspective cross-sectional view with a partial cutout illustrating an exemplary embodiment of an apparatus for detecting a microorganism or micro-particle in real time according to the present invention.

Referring to FIG. 2, an atmosphere 310 including a microorganism or micro-particle is introduced into the apparatus for detecting a microorganism or micro-particle in real time via a filter module 300. The filter module 300 filters out a microorganism or micro-particle which is larger than a predetermined size from the atmosphere 310. In exemplary embodiments, the filter module 300 which filters the microorganism or micro-particle may include a pore size between about 0.5 μm and about 10 μm.

The atmosphere 310 which passes through the filter module 300 makes contact with the collection module 100. The atmosphere 310 is condensed to a droplet 111 by a condensation element unit 110, and a droplet stream is generated by passing through a collection channel unit 120. The generated droplet stream is introduced into the sensing module 200. The microorganism or micro-particle is detected and counted in the sensing module 200. The collection module 100 is described in more detail with reference to FIGS. 3A and 3B.

FIGS. 3A and 3B are a cross-sectional side view and a cross-sectional front view, respectively, of an exemplary embodiment of a collection module 100 according to the present invention.

Referring to FIGS. 3A and 3B, the atmosphere 310 which passes through the filter module 300 is introduced into a collection channel unit 120, which faces a condensation element unit 110. The atmosphere 310 is cooled in the condensation element unit 110, condensed in the collection channel unit 120 and thereby forms a droplet 111. Also, the droplet 111 flocculates and thereby generates a droplet stream. The droplet stream is introduced into a flow unit 210 of the sensing module 200.

In exemplary embodiments, the condensation element unit 110 may be a Peltier element. A DC current is passed through two dissimilar metals which are connected to each other at two junctions, and the DC current drives a transfer of heat from one junction to the other. Specifically, one junction cools off while the other heats up, which is used in the Peltier element. The Peltier element according to the current exemplary embodiment includes a heat absorption unit, a heating unit and a PNP type or a NPN type semiconductor device disposed between the heat absorption unit and the heating unit. In exemplary embodiments, the PNP type semiconductor includes a n-type semiconductor disposed between two p-type semiconductors. In further exemplary embodiments, the NPN type semiconductor includes a p-type semiconductor disposed between two n-type semiconductors.

Temperature decreases in the heat absorption unit, and increases in the heat generation unit due to a current supply. The heat absorption unit faces the collection channel unit 120, and thus the atmosphere 310, which is introduced into the collection channel unit 120, is cooled and condensed by the heat absorption unit. In exemplary embodiments, the condensation element unit 110 may further include a temperature control unit, which is not illustrated, to control a temperature for condensing the atmosphere 310. Specifically, the atmosphere 310 is efficiently condensed by the heat absorption unit of the condensation element unit 110, and thereby forms the droplet 111. The droplet 111 is condensed, and thereby generates a droplet stream. The droplet stream is introduced into the flow unit 210 of the sensing module 200.

The collection channel unit 120 includes a channel unit 120 where a hydrophilic member 121 is formed. The hydrophilic member 121 is in a vein structure to help a collection of water for generating the droplet 111 and to help the droplet stream to flow more smoothly.

In exemplary embodiments, the hydrophilic member 121 includes at least one of a poly(hydroxy ethyl methacrylate), a poly(N-vinyl pyrrolidone), a polyethylene oxide, a polyproplyene oxide, a poly(N-isoproply acrylamide) polyethylene terephthalate, a polymethyl methacrylate, a poly(acrylic acid), a poly(vinyl alcohol), a poly(dimethyl siloxane), an epoxy resin and a combination including at least one of the foregoing. In further exemplary embodiments, the hydrophilic member 121 includes a pattern of vein structure. The pattern of vein structure enables the droplet stream to efficiently flow into the flow unit 210 of the sensing module 200. In exemplary embodiments, the pattern of vein structure may be formed using a disclosed lithography method, an imprint method, or a self-assembly method.

In exemplary embodiments, the collection channel unit 120 may further include a hydrophobic member 122 which is located around the hydrophilic member 121. The hydrophobic member 122 prevents the droplet 111 and the droplet stream from diffusing, and enables the droplet stream to flow more smoothly via the collection channel unit 120. In the current exemplary embodiment, the collection channel unit 120 is comprised of a plurality of channels including the hydrophilic member 121. Accordingly, a microorganism or micro-particle which is adhered to the droplet 111 may be easily detected and counted in a sensing module 200. In exemplary embodiments, the hydrophobic member 122 includes at least one of an ethylene-tetrafluoroethylene copolymer, a poly(chlorotrifluoroethylene) resin, an ethylene-chlorotrifluoroethylene copolymer, a tetrafluoroethylene-perfluoroalkylvinylether copolymer, a tetrafluoroethylene-hexafluoropropylene copolymer and a combination including at least one of the foregoing. However, the hydrophobic member 122 according to the current exemplary embodiment is not limited to the above-described examples, and may be applied to the above-described materials including a chemical structure such as a two-tri-multi block, a grafting, a star shape, a dendriner, a comb shape, a hyperbranch, or an interpenetrating polymer network (“IPN”). Also, in further exemplary embodiments, the above-described materials may further include a porous structure with a pore size from about 0.01 μm to about 1000 μm. When the above-described materials include the porous structure, a hydrophobicity increases. Accordingly, the droplet 111 and the droplet stream may be more efficiently prevented from diffusing, and the droplet stream may flow more smoothly via the collection channel unit 120.

In exemplary embodiments, the atmosphere 310 which is not condensed may be exhausted via a predetermined outlet, as illustrated in FIG. 3A.

Also, in exemplary embodiments, the droplet stream, which is formed in the plurality of channels, may form a branch stream. The branch stream of the droplet stream is combined in a central channel, and thereby may form a larger droplet stream. The collection channel unit 120 according to the current exemplary embodiment of the present invention is not limited to the pattern of vein structure, and may be applied to other structures which may efficiently gather the droplet stream.

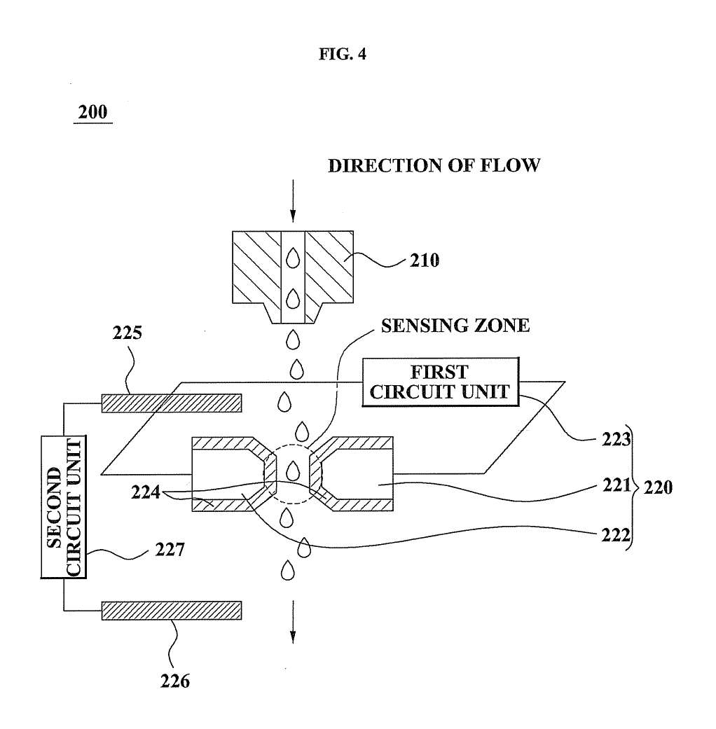

Referring again to FIG. 2, the droplet stream to which the microorganism or micro-particle in the atmosphere 310 adheres to is introduced into the sensing module 200. The introduced microorganism or micro-particle is detected and counted in the sensing module 200. The sensing module 200 is described in more detail with reference to FIG. 4.

FIG. 4 is a cross-sectional view of an exemplary embodiment of a sensing module 200 according to the present invention.

Referring to FIG. 4, the sensing module 200 includes a Coulter counter 220. A droplet stream is introduced to the Coulter counter 220 which detects and counts a microorganism or micro-particle which is adhered to the droplet stream. The droplet stream is introduced to the Coulter counter 220 in the direction corresponding to the direction of flow, as illustrated in FIG. 4. In exemplary embodiments, the sensing module 200 may include a flow unit to efficiently introduce the droplet stream to which the microorganism or micro-particle adheres. Also, in further exemplary embodiments, the sensing module 200 may further include a stream control unit 210 which controls a flow velocity or a flow rate of the droplet stream to more efficiently detect and count the microorganism or micro-particle. In exemplary embodiments, the stream control unit 210 may include a configuration as illustrated in FIG. 4 in order to control the flow velocity or the flow rate of the introduced droplet stream. However, the stream control unit 210 is not limited to the configuration as illustrated in FIG. 4, and the stream control unit 210 may be applied in various configurations for controlling the flow velocity or the flow rate of the droplet stream.

The droplet stream, of which the flow velocity or the flow rate is controlled, is introduced into the Coulter counter 220, and the microorganism or micro-particle is detected and counted via the Coulter counter 220. The Coulter counter 220 has been used as a cell counter which measures a size and a number of red blood cells. In the Coulter counter 220, two electrodes, which are separately disposed in an electrolyte, generate a current. In this instance, an orifice is located between the two electrodes. When dispersed micro-particles in the electrolyte pass through the orifice, the micro-particles, e.g., non-conductive materials, occupy an inner space of the orifice which has been previously occupied by the electrolyte. Accordingly, a volume which has been occupied by the electrolyte is reduced, and thus an impedance increases. When the micro-particles are conductive materials, an assumption in which the micro-particles in the orifice include an infinite resistance is valid, since a contact surface between the micro-particles and the electrolyte interferes with a current flow, which is introduced to the micro-particles, due to a polarization effect. An orifice area sensing an impedance change is referred to as a sensing zone. A pair of electrodes in the orifice senses a relatively small change in impedance.

The Coulter counter 220 includes two electrodes, a cathode 225 and an anode 226, through which a predetermined amount of current flows. The cathode 225 and the anode 226 may be separated in exemplary embodiments. In the current exemplary embodiment, the orifice is between the cathode 225 and the anode 226. The Coulter counter includes an electrode unit and a first circuit unit 223. The electrode unit includes a pair of electrodes 221 and 222. The pair of electrodes 221 and 222 forms the sensing zone. The first circuit unit 223 measures an impedance change which is sensed when the microorganism or micro-particle passes through the sensing zone. Also, in exemplary embodiments, the Coulter counter 220 may further include a second circuit unit 227 which measures an impedance change between electrolytes of two compartments. The two compartments are separated by the orifice through which the microorganism or micro-particle passes. That is, in exemplary embodiments, the two compartments are separated by the pair of electrodes 221 and 222.

Also, a photocatalytic material 224 is coated on a surface of the pair of electrodes 221 and 222, which helps a sterilization of the microorganism or micro-particle by a sterilization module 400 which will now be described. In exemplary embodiments, the photocatalytic material 224 may be comprised of any one of titanium dioxides (“TiO₂”), which are doped in any one of titanium dioxide (“TiO₂”), fluorine (F), iron (Fe), vanadium (V) and nitrogen (N). Also, zinc oxide (“ZnO”), cadmium sulfide (“CdS”), zirconium oxide (“ZrO₂”), tin oxide (“SnO₂”), vanadium trioxide (“V₂O₃”), tungsten oxide (“WO₂”) and strontium titanate (“SrTiO₃”) may be used as the photocatalytic material 224 in exemplary embodiments. In the current exemplary embodiment, the sterilization module 400 includes an ultraviolet ray. However, the present invention is not limited thereto.

In an exemplary embodiment, the first circuit unit 223 may further include a predetermined amplifier unit. The amplifier unit easily measures a change of a current pulse in the pair of electrodes 221 and 222 by measuring an impedance change when the microorganism or micro-particle passes through the sensing zone. As the microorganism or micro-particle passes through the sensing zone, the relatively small impedance value increases. The change of the current pulse is stored as data, and the microorganism or micro-particle is counted. A size of the pulse is dependent on a volume, e.g., a diameter or a size, of the micro-particle. Accordingly, in exemplary embodiments, the size of the microorganism or micro-particle may be ascertained by a measured pulse size. The photocatalytic material 224, coated on the surface of the pair of electrodes 221 and 222, supports the sterilization module 400. Also, in exemplary embodiments, a type of the microorganism or micro-particle may be distinguished depending on a type of the photocatalytic material 224, since a duration time of the current change pulse, a pulse pattern, or a noise pattern depends on the type of the photocatalytic material 224. The duration time of the current change pulse refers to a period of time when the microorganism or micro-particle is in the sensing zone.

Accordingly, a database of a quantitative and a qualitative result of a current change pulse waveform, which is measured in the sensing module 200, is built. Also, the database and the measured current change pulse waveform are analyzed. In an exemplary embodiment, a result of the analysis may be outputted to a predetermined display module and transmitted to another module requiring the result of the analysis.

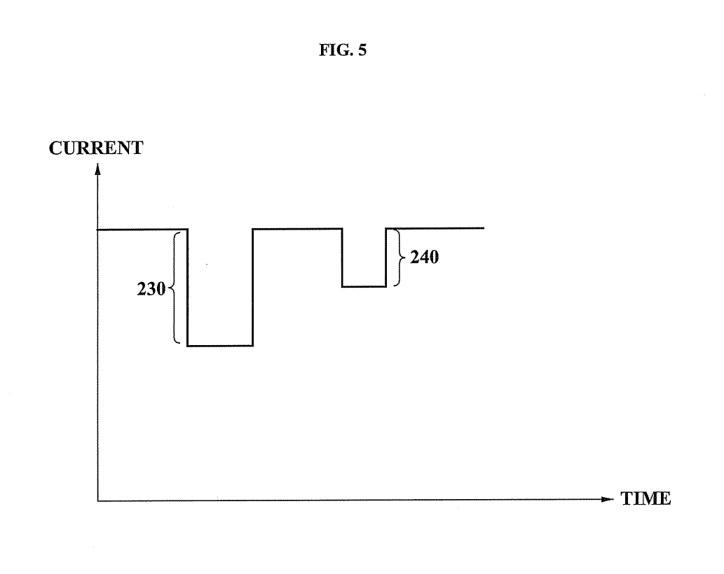

FIG. 5 is a pulse waveform diagram of a current change measured in an exemplary embodiment of a sensing module 200 according to the present invention.

Referring to FIG. 5, the first circuit unit 223 and a second circuit unit 227 measures an impedance change which is sensed when a microorganism or micro-particle passes through a sensing zone. Also, the first circuit unit 223 and the second circuit unit 227 measure a change of a current pulse by amplifying a change of a small impedance through a predetermined amplifier unit. A variety of current change pulses 230 and 240 are generated depending on a type and a size of the microorganism or micro-particle passing through the sensing zone. In exemplary embodiments, the current change pulses 230 and 240 are stored as data, and thus the microorganism or micro-particle may thereby be counted. In further exemplary embodiments, a density of the microorganism or micro-particle may be measured by using a number of the measured current change pulses. A size of the pulse is dependent on a volume, e.g., a diameter or a size, of the micro-particle. Accordingly, in an exemplary embodiment, the size of the microorganism or micro-particle may be ascertained by the measured pulse size. The photocatalytic material 224, coated on a surface of a pair of electrodes 221 and 222, supports a sterilization module 400. In exemplary embodiments, a type of the microorganism or micro-particle may be distinguished depending on a type of the photocatalytic material 224, since a duration time of the current change pulse, a pulse pattern, or a noise pattern depends on the type of the photocatalytic material 224. The duration time of the current change pulse refers to a period of time when the microorganism or micro-particle is in the sensing zone.

Referring again to FIG. 2, the microorganism or micro-particle, detected and counted by the sensing module 200, is sterilized by the sterilization module 400. The current exemplary embodiment of the present invention is not limited to an ultraviolet (“UV”) lamp, and may be applied with an apparatus which may sterilize the microorganism or micro-particle.

A droplet stream, which passes the sterilization module 400, is vaporized by a drain module 500 by heating, and may be discharged via an outlet 520 of the drain module 500. In an exemplary embodiment, a heating coil 510 may not wind around the outlet 520. Specifically, a configuration, which may efficiently heat the droplet stream passing the sterilization module 400, may be applied in an exemplary embodiment of the present invention.

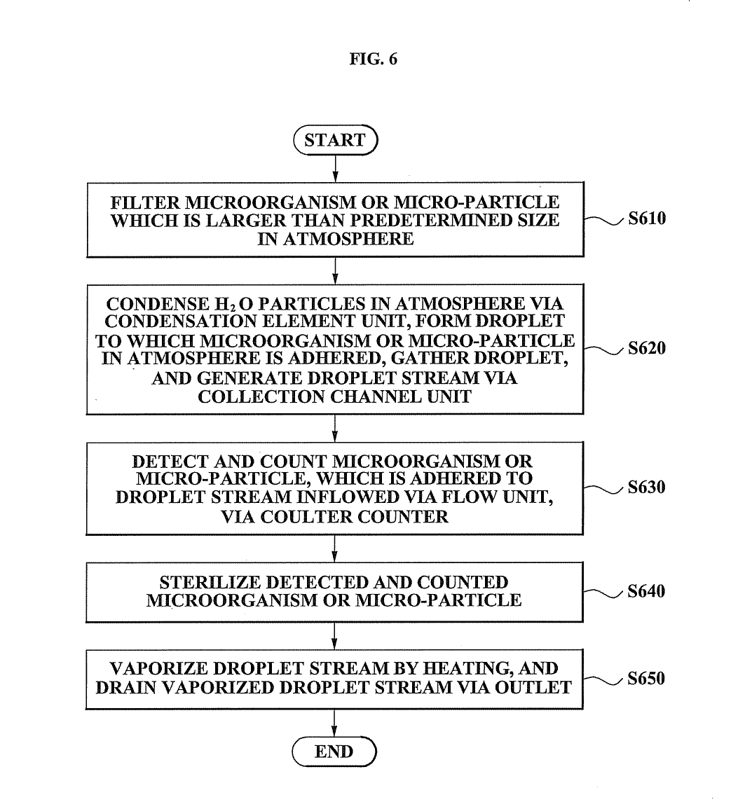

FIG. 6 is a flowchart illustrating an exemplary embodiment of a method of detecting a microorganism or micro-particle in real time according to the present invention.

Referring to FIG. 6, a microorganism or micro-particle which is larger than a predetermined size in an atmosphere 310 is filtered out in operation S610. In the current exemplary embodiment, a microorganism or micro-particle between about 0.5 μm and about 10 μm may be filtered.

In operation S620, water (“H₂O”) particles in an atmosphere 310 is condensed in a condensation element unit 110, and a droplet 111 to which a microorganism or micro-particle in the atmosphere 310 adheres to is formed in a condensation element unit 110. The droplet 111 is gathered via a collection channel unit 120 and a droplet stream is generated. The collection channel unit 120 is comprised of a hydrophilic material 121. The collection channel unit 120 includes a cone-shape channel whose channel radius becomes gradually narrower in a direction where the droplet stream flows. Also, the condensation element unit 110 is a Peltier element.

In operation S630, the droplet stream is introduced via a predetermined inflow unit, and the microorganism or micro-particle which adheres to the introduced droplet stream is detected and counted via a Coulter counter. In exemplary embodiments, when detecting and counting the microorganism or micro-particle which is adhered to the introduced droplet stream, the droplet stream may be controlled by controlling any one of a flow velocity and a flow rate of the droplet stream. The Coulter counter includes two electrodes through which a predetermined amount of current flows. In exemplary embodiments, the two electrodes are a negative pole and a positive pole, and may be separated. In the current exemplary embodiment, an orifice is disposed between the two electrodes. The Coulter counter includes an electrode unit and a first circuit unit. The electrode unit includes a pair of electrodes. The pair of electrodes forms a sensing zone. The first circuit unit measures the impedance change which is sensed when the microorganism or micro-particle passes through the sensing zone. Also, in exemplary embodiments, the Coulter counter may further include a second circuit unit which measures an impedance change between electrolytes of two compartments. The two compartments are separated by the orifice through which the microorganism or micro-particle passes. Also, a photocatalytic material is coated on a surface of the pair of electrodes, which helps a sterilization of the microorganism or micro-particle with a sterilization module 400 which will be described. In exemplary embodiments, the photocatalytic material may be comprised of any one of titanium dioxides (“TiO₂”), which are doped in any one of titanium dioxide (“TiO₂”), fluorine (F), iron (Fe), vanadium (V), and nitrogen (N). Also, zinc oxide (“ZnO”), cadmium sulfide (“CdS”), zirconium oxide (“ZrO₂”), tin oxide (“SnO₂”), vanadium trioxide (“V₂O₃”), tungsten oxide (“WO₂”), and strontium titanate (“SrTiO₃”) may be used as the photocatalytic material in a particular condition. The photocatalytic material, coated on the surface of the pair of electrodes, supports the sterilization module 400. In exemplary embodiments, a type of the microorganism or micro-particle may be distinguished depending on a type of the photocatalytic material, since a duration time of the current change pulse, a pulse pattern, or a noise pattern depends on the type of the photocatalytic material. The duration time of the current change pulse refers to a period of time when the microorganism or micro-particle is in the sensing zone.

In operation S640, the detected and counted microorganism or micro-particle is sterilized. In operation S650, the droplet stream is vaporized by heating, and the vaporized droplet stream is drained via an outlet.

The method of detecting a microorganism or micro-particle in real time according to the above-described exemplary embodiments of the present invention may be recorded in computer-readable media including program instructions in order to implement various operations embodied by a computer. In exemplary embodiments, the media may also include, alone or in combination with the program instructions, data files, data structures and the like. In further exemplary embodiments, the media and program instructions may be those specially designed and constructed for the purposes of the present invention, or they may be of the type well-known and available to those having ordinary skill in the computer software arts. Exemplary embodiments of computer-readable media include magnetic media such as hard disks, floppy disks and magnetic tape; optical media such as CD ROM disks and DVD; magneto-optical media such as optical disks; and hardware devices which are specially configured to store and perform program instructions, such as read-only memory (“ROM”), random access memory (“RAM”), flash memory and the like. In exemplary embodiments, the media may also be a transmission medium such as optical or metallic lines, wave guides and the like, including a carrier wave which transmit signals specifying the program instructions, data structures and the like. Exemplary embodiments of program instructions include both machine code, such as produced by a compiler, and files containing higher level code which may be executed by the computer using an interpreter. In exemplary embodiments, the described hardware devices may be configured to act as one or more software modules in order to perform the operations of the above-described exemplary embodiments of the present invention.

According to the above-described exemplary embodiments of the present invention, an apparatus and method of detecting a microorganism or micro-particle in real time does not require a complex and expensive apparatus.

Also, according to the above-described exemplary embodiments of the present invention, an apparatus and method of detecting a microorganism or micro-particle in real time does not require an additional conversion of an electrical signal.

Also, according to the above-described exemplary embodiments of the present invention, an apparatus and method of detecting a microorganism or micro-particle in real time is based on a wet measurement and does not require an additional apparatus for providing water.

Although a few exemplary embodiments of the present invention have been shown and described, the present invention is not limited to the described exemplary embodiments. Instead, it would be appreciated by those of ordinary still in the art that changes may be made to these exemplary embodiments without departing from the principles and spirit of the present invention, the scope of which is defined by the claims and their equivalents.

What is claimed is:

1. An apparatus for detecting a microorganism or micro-particle in real time, the apparatus comprising:
a collection module comprising a condensation element unit which condenses water particles in an atmosphere and which forms a droplet to which a microorganism or micro-particle in the atmosphere is adhered to, and a collection channel unit which gathers the droplet and generates a droplet stream; and
a sensing module comprising a counting module, the sensing module in fluid communication with the droplet stream the sensing module detects and counts the microorganism or micro-particle which is adhered to the droplet stream.

2. The apparatus of claim 1, further comprising:
a filter module which filters a microorganism or micro-particle which is larger than a certain size from the atmosphere, the filter module guides the filtered microorganism or micro-particle to the collection module.

3. The apparatus of claim 2, wherein the microorganism or micro-particle allowed to pass through the filter module is between about 0.5 μm and about 10 μm.

4. The apparatus of claim 1, further comprising:
a sterilization module which sterilizes the detected and counted microorganism or micro-particle.

5. The apparatus of claim 4, further comprising:
a drain module which drains a vaporized droplet stream via an outlet, the droplet stream being vaporized by heating.

6. The apparatus of claim 1, wherein the condensation element unit is a Peltier element.

7. The apparatus of claim 1, wherein the collection channel unit includes a pattern where a hydrophilic member is formed in a hydrophobic member, the hydrophilic member being in a vein structure.

8. The apparatus of claim 7, wherein the hydrophobic member comprises at least one of an ethylene-tetrafluoroethylene copolymer, a poly(chlorotrifluoroethylene) resin, an ethylene-chlorotrifluoroethylene copolymer, a tetrafluoroethylene-perfluoroalkylvinylether copolymer, a tetrafluoroethylene-hexafluoropropylene copolymer and a combination including at least one of the foregoing.

9. The apparatus of claim 7, wherein the hydrophilic member comprises at least one of a poly(hydroxy ethyl methacrylate), a poly(N-vinyl pyrrolidone), a polyethylene oxide, a polyproplyene oxide, a poly(N-isoproply acrylamide) polyethylene terephthalate, a polymethyl methacrylate, a poly(acrylic acid), a poly(vinyl alcohol), a poly(dimethyl siloxane), an epoxy resin and a combination including at least one of the foregoing.

10. The apparatus of claim 1, wherein the sensing module further comprises a stream control unit which controls at least one of a flow velocity and a flow rate of the droplet stream.

11. The apparatus of claim 1, wherein the sensing module comprises a Coulter counter.

12. The apparatus of claim 1, wherein the sensing module comprises a first circuit and a second circuit, the first circuit measures an impedance change of the droplet stream and the second circuit measures an impedance change between at least two compartments.

13. A method of detecting a microorganism or micro-particle in real time, the method comprising:
condensing water particles in an atmosphere via a condensation element unit;
forming a droplet from the condensed water particles to which a microorganism or micro-particle in the atmosphere is adhered to;
gathering the droplet and generating a droplet stream via a collection channel unit, the collection channel unit comprising a hydrophilic material; and
introducing the droplet stream via a predetermined inflow unit, and detecting and counting the microorganism or micro-particle which is adhered to the introduced droplet stream via a Coulter counter.

14. The method of claim 13, further comprising:
filtering a microorganism or micro-particle which is larger than a predetermined size from the atmosphere.

15. The method of claim 13, further comprising:
sterilizing the detected and counted microorganism or micro-particle.

16. The method of claim 15, further comprising:
draining a vaporized droplet stream via an outlet, the droplet stream being vaporized by heating.

17. The method of claim 13, wherein the condensation element unit is a Peltier element.

18. The method of claim 13, wherein the collection channel unit comprises a cone-shape channel having a channel radius which becomes gradually narrower in a direction in which the droplet stream flows.

19. The method of claim 13, wherein the detecting and counting further comprises:
controlling the droplet stream by controlling at least one of a flow velocity and a flow rate of the droplet stream.

20. At least one medium comprising computer readable instructions implementing a method of detecting a microorganism or micro-particle in real time, the method comprising:
condensing water particles in an atmosphere via a collection channel unit;
forming a droplet with a condensation element unit, a microorganism or micro-particle in the atmosphere adheres to the droplet;
gathering the droplet and generating a droplet stream, the collection channel unit comprising a hydrophilic material;
introducing the droplet stream via a predetermined flow unit; and
detecting and counting the microorganism or micro-particle which adheres to droplet stream via a Coulter counter.

21. A collection module comprising;
a condensation element unit which condenses water particles in an atmosphere and which forms a droplet to which a microorganism or micro-particle in the atmosphere is adhered to; and
a collection channel unit which gathers the droplet and generates a droplet stream.
